(19) United States
(12) Patent Application Publication
(10) Pub. No.: US 2007/0021809 A1
(43) Pub. Date: Jan. 25, 2007

(54) Vagina and vulva cooling and heating device

(76) Inventors: Pamela S. Cole, Hopkins, MN (US); Jack W. Smith, Minnetonka, MN (US); Curtis D. Kinghorn, Lino Lakes, MN (US)

Correspondence Address: Curtis D. Kinghorn;Sirius Strategic, LLC, 6769 W. Shadow Lake Dr., Lino Lakes, MN, 55014, US

(21) Appl. No.: 11/187,320

(22) Filed: Jul. 22, 2005

(51) Int. Cl. A61F 7/12

(52) U.S. Cl. 607/113

(57) ABSTRACT

The present invention is a device and therapeutic method that provides topical cooling or warming to a user's vagina or vulva as a therapeutic benefit to and relief from maladies of these areas. The device includes a protrusion that is placed into the user's vagina and a base that contacts the female genitals around the vagina. The device is cooled or warmed prior to or during use. The method includes the use of the device to provide a therapeutic effect and relief from maladies of a user's vagina or vulva.

### BACKGROUND OF THE INVENTION

1. Field of the Invention

The present invention relates to the field of treatment of feminine disorders and more particularly to the treatment of the symptoms of inflammation or irritation to a woman's genitals.

2. Description of Related Art

There are many conditions that may affect a woman's vagina causing discomfort. A broad category of such afflictions is vaginitis which is defined as an inflammation of the vagina characterized by discharge, odor, irritation and itching or a combination of these. Vaginitis is often caused by infections. The most common vaginal infections are bacterial vaginoisis, trichomoniasis and vaginal yeast infection or candidiasis. Symptoms of vaginitis, particularly with trichomoniasis and vaginal yeast infection, include irritation and itching of the female genital area including itching, burning and irritation of the vagina.

Vaginitis is one of the most common problems in clinical medicine and accounts for more than 10 million office visits each year. (Kent, Howard L. “Epidemiology of Vaginitis,” American Journal of Obstetrics and Gynecology 165, no. 4, part 2 (October 1991): 1168-76.) Vaginitis is reported to be the most common reason for a patient to visit her obstetrician-gynecologist. Id. A study conducted by B. Foxman, R. Barlow, H. D'Arcy, B Gillespie and JD Sobel at the Department of Epidemiology, University of Michigan School of Public Health Studies (Foxman B, Barlow R, D'Arcy H, Gillespie B, Sobel JD, “Candida vaginitis: self-reported incidence and associated costs,” Department of Epidemiology, University of Michigan School of Public Health, Ann Arbor 48109-2029, USA) showed that a total of 6.5 percent of women older than 18 years reported a least one episode of presumed vaginitis during the previous 2 months. That same study showed that 8.0 percent of the women reported having four or more episodes of vaginitis in a 1-year period. Although these women accounted for only 8.0 percent of the sample, they accounted for 37.2 percent of women reporting episodes.

Vaginitis is found in 28% of women attending sexually transmitted disease (STD) clinics; sexually active women are at an increased risk for vaginitis because the presence of semen in the vagina may raise the pH and thereby allow for a proliferation of pathogenic anaerobic bacteria. Epidemiology of Vaginitis at Id. Candidiasis is the second most frequent vaginal infection in the United States and the primary vaginal infection in Europe. Id. The late Herman Gardner put the human toll of vaginitis in perspective when he stated, “Vaginitis must cause more unhappiness on earth than any other gynecologic disease. In addition to the many physical and emotional problems associated with vaginitis, the economic loss involved is of astronomic proportions.” (Kaufman RN, Freidrich EG, Gardner HL. Benign diseases of the vulva and vagina. 3rd ed. Chicago: Year Book Medical Publishers, 189:361-418.)

Treatment for the various presentations of vaginitis usually includes the application of antibiotics or acidophilus or both, usually orally. Also, where the inflammation or irritation is present in the vagina, antifungal medications are often used. These antifungal treatments come in the form of creams, tablets or suppositories such as boric capsules. These treatments are typically applied several times a day for several weeks. A disadvantage of these treatments is that relief from the vaginitis symptoms takes several days or weeks to occur.

Some women find relief from the symptoms of vaginitis from the application of an acidophilus-containing yogurt douche. This treatment is typically applied daily for a few days or weeks depending on the severity of the infection. This treatment has at least the disadvantage of being somewhat messy and again taking a relatively long time to be effective and provide relief.

Also, women who have a yeast infection (or are predisposed to such infections) are often advised to limit their intake of sugar, fruit juices and refined carbohydrates. For persistent or recurrent infections, some doctors recommend that fruit also be avoided. While these lifestyle changes may ultimately be effective in treating or reducing the incidence of vaginitis, these benefits take a long time to come into effect after the lifestyle changes are made.

In any event, there is often a delay from the time treatment is begun until relief, particularly significant relief, from the symptoms is found. As a result, women with vaginitis, even after receiving treatment, continue to experience the symptoms of irritation, itching and burning of the female genitals generally and the vagina specifically.

Another broad category of conditions that may affect a woman's genitals and vagina is inflamed vaginal and vulvar tissue resulting from trauma or irritation. This inflammation may result from trauma or irritation from sexual activity especially due to increased sexual activity, sexual activity with inadequate vaginal lubrication especially in post menopausal women and sexual activity for many post menopausal women who have decreased elasticity in their vaginal tissues. This inflammation may also be present in post-partum women as a result of the trauma or irritation from the childbirth process. There are other conditions or reasons that a woman might experience vaginal inflammation. The treatments for vaginal inflammation resulting from other conditions or reasons mirror the treatments described above.

Treatments for this inflammation, whatever the source, include the application of topical creams or lotions and tablets or suppositories and the application of cool or warm compresses. These treatments are typically applied several times a day for several weeks. A disadvantage of these treatments again is that little significant relief occurs immediately and ultimate relief from the symptoms takes several days or weeks to occur.

The treatments described above in connection with the variety of maladies of the female genitals and vagina typically have the further disadvantage of often requiring the user to obtain a prescription from her physician and then getting the prescription filled at a pharmacist before being able to apply the therapy. Both of these actions take time and money. Often, the woman suffering from these afflictions seeks more immediate relief, especially relief that is cheaper and easier to obtain. Consequently, there exists a need for a cheap, easy, accessible and reliable treatment for these maladies of the vagina and vulva.

### SUMMARY OF THE INVENTION

The present invention is a device and therapeutic method that provides topical cooling or heating to a user's vagina or vulva as a therapeutic benefit to and relief from maladies of these areas. The device includes a protrusion that is placed into the user's vagina and a base that contacts the female genitals around the vagina. The device is cooled or heated prior to or during use. The method includes the use of the device to provide a therapeutic effect and relief from maladies of a user's vagina or vulva.

It is therefore an object of the invention in one embodiment to provide topical cooling or heating to a user's vagina or vulva or both.

It is an object of the invention in one embodiment to provide a method of treating inflammation or irritation to a woman's genitals.

It is an object of the invention in one embodiment to provide a method of treating vaginitis.

It is an object of the invention in one embodiment to provide a method of treating inflamed vaginal and vulvar tissue resulting from trauma or irritation.

It is an object of the invention in one embodiment to provide a method of treating inflamed vaginal and vulvar tissue resulting from sexual activity with inadequate vaginal lubrication especially in post menopausal women and from sexual activity for post menopausal women who have decreased elasticity in their vaginal tissues.

It is an object of the invention in one embodiment to provide a method of treating inflamed vaginal and vulvar tissue resulting from increased sexual activity.

It is an object of the invention in one embodiment to provide a method of treating inflamed vaginal and vulvar tissue in post-partum women resulting from trauma or irritation from the childbirth process.

It is an object of the invention in one embodiment to provide a method of treating inflamed vaginal and vulvar tissue in non-human animals resulting from trauma or irritation.

These and other objects of the invention will be clear from the description of the invention given herein.

### BRIEF DESCRIPTION OF THE DRAWINGS

FIG. 1 is a side cross-sectional view of the female genitals.

FIG. 2 is a perspective view of one embodiment of the present invention.

FIG. 3 is a side view of the invention of FIG. 2.

FIG. 4 is a top view of the invention of FIG. 2.

FIG. 5 is a bottom view of the invention of FIG. 2.

FIG. 6 is an end view of the invention of FIG. 2.

FIG. 7 is a perspective cutaway view of another embodiment of the present invention.

FIG. 8 is a perspective cutaway view of another embodiment of the present invention.

FIG. 9 is a perspective cutaway view of another embodiment of the present invention.

FIG. 10 is a perspective cutaway view of another embodiment of the present invention.

FIG. 11 is a perspective cutaway view of another embodiment of the present invention.

FIG. 12 is a perspective cutaway view of another embodiment of the present invention.

FIG. 13 is a perspective cutaway view of another embodiment of the present invention.

FIG. 14 is a perspective cutaway view of another embodiment of the present invention.

FIG. 15 is a perspective cutaway view of another embodiment of the present invention.

FIG. 16 is a perspective cutaway view of another embodiment of the present invention.

FIG. 17 is a schematic drawing of a Peltier circuit.

FIG. 18 is a perspective view of an embodiment of the invention incorporating a Peltier module.

FIG. 19 is a perspective cutaway view of another embodiment of the invention incorporating a Peltier module.

FIG. 20 is a side cutaway view of another embodiment of the invention incorporating a Peltier module.

FIG. 21 is a side cutaway view of another embodiment of the invention incorporating a Peltier module.

FIG. 22 is a side cutaway view of another embodiment of the invention incorporating a Peltier module.

FIG. 23 is a perspective view of a container of the present invention.

FIG. 24 is a schematic view of a kit of the present invention.

FIG. 25 is a side cross-sectional view of the present invention in use in contact with a female user's genitals.

FIG. 26 is a perspective view of an embodiment of the invention having only a projection.

FIG. 27 is a side cross-sectional view of the invention of FIG. 26 in use in contact with a female user's genitals.

FIG. 28 is a side view of an embodiment of the invention having only a base.

FIG. 29 is a side cross-sectional view of the invention of FIG. 28 in use in contact with a female user's genitals.

FIG. 30 is a side view of the invention of FIG. 2 with exemplary dimensions added.

FIG. 31 is a bottom view of the invention of FIG. 2 with exemplary dimensions added.

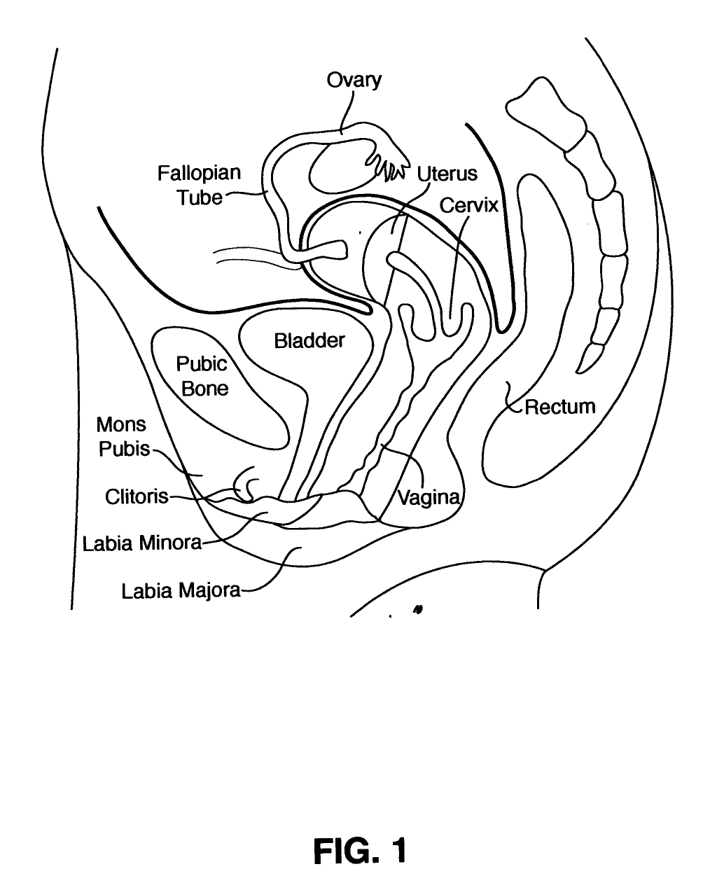

### DETAILED DESCRIPTION OF THE INVENTION

The invention is shown in the Figures generally labeled 10. The device comprises an elongated projection 12 attached to a base 14. Throughout the description, like reference numbers, wherever referred to and regardless of the embodiment particularly described in association therewith, unless specifically stated otherwise, refer to like elements. The properties, characteristics or attributes of such elements described in connection with a specific embodiment or a variant within a specific embodiment apply to similarly referenced elements even if used in connection with alternate embodiments or variants unless specifically stated otherwise.

The projection 12 is a protrusion that has a distal end 16, a proximal end 18 that is attached to base 14 and a projection outer surface 20. Projection 12 is preferably hourglass shaped so that the diameter “D” of projection 12 between distal end 16 and proximal end 18 at narrowing 22 is somewhat less than the diameters of either distal end 16 or proximal end 18.

Although the projection 12 is preferably hourglass shaped, it is not required to be. In fact, projection 12 may have a constant diameter or increasing or decreasing diameter going from the distal end 16 to the proximal end 18. Further, projection 12 may be straight or curved as desired to give the most comfortable and effective fit between the projection 12 and the walls of the vagina when the device 10 is in operation as will be described hereafter. The distal end 16 of projection 12 is preferably rounded to facilitate its insertion into the user's vagina.

As stated, the projection 12 in operation will be inserted into the user's vagina. In the embodiment of projection 12 having a narrowing 22, narrowing 22 is located so that that narrowing 22 will be located at the ringlet muscles of the vagina when the projection 12 is inserted into the vagina. This configuration allows the surface of projection 12 to have maximal contact with the tissue of the vagina on either side of the “ringlet muscles” of the vagina.

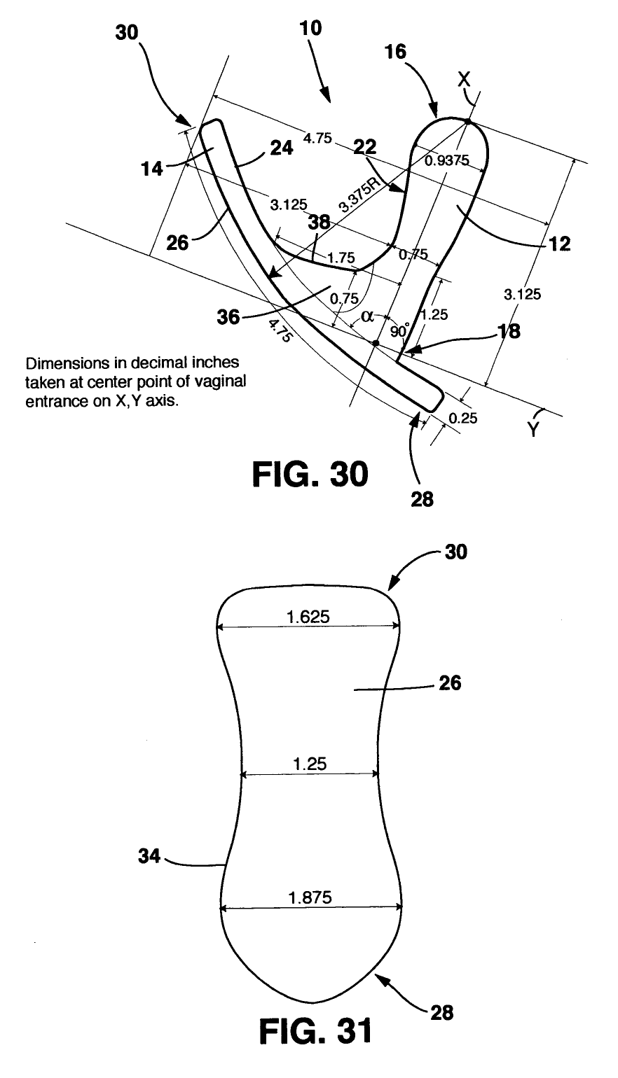

It is well understood that women have varying anatomy with respect to aspects of their genitals including vaginal length. Some of this anatomy statistically depends on the size and age of the woman as well as the woman's race. As a result, the length of the projection 12 and the location of the narrowing 22 or both that will work best for one woman will not necessarily work well for another. Because of this, it is anticipated that the device could be offered in a variety of dimensions of projection 12 and location of narrowing 22 (FIG. 30). However, it is believed that the typical length “L” of projection 12 should be about 3.125 inches and the typical location of the narrowest part of narrowing 22 should be about 1.25 inches from the proximal end 18. Of course, the length L of projection 12, as described in one embodiment hereafter, could be a short as being non-existent. Conversely, it is anticipated that a good representative range of lengths “L” for projection 12 to cover most women regardless of their race or size would be approximately ⅔ of the preferred length (approximately 2.083 inches) to 1.5 times the preferred length (approximately 4.688 inches) with the location of narrowing 22 located at any point between these exemplary endpoints.

These dimensions for length L have been given as exemplary and are not intended to be limiting. Instead, the maximum length L for the projection 12 could be as long as desired to provide comfort to the user but not so long as to provide discomfort for, among other reasons, the distal end 16 of the projection contacting the user's cervix. Likewise, the location of narrowing 22 should ideally be located at a point most comfortable to the user and may vary from woman to woman along nearly the entire length L of projection 12.

Also, it is clear that because women have varying anatomy, projections 12 of varying diameter as part of the device 10 in use will be more or less comfortable to the user. As a result, it is also anticipated that the device 10 may be offered with a variety of diameters for projection 12 as well. An example of a typical diameter “D” of projection 12 is about 0.9375 inches with a typical diameter of the narrowing 22 of about 0.75 inches.

Once again, the diameter D of projection 12 could be as small as being close to non-existent. Conversely, it is anticipated that a good representative range of diameters D for projection 12 to cover most women regardless of their race or size would be approximately ⅔ of the preferred diameter (approximately 0.625 inches) to 1.5 times the preferred diameter (approximately 1.406 inches) with the corresponding diameter of narrowing 22 ranging also from ⅔ of the preferred diameter (approximately 0.5 inches) to 1.5 times the preferred diameter (approximately 1.125 inches).

These dimensions for diameter D have been given as exemplary and are not intended to be limiting. Instead, the maximum thickness D for the projection 12 could be as wide as desired to provide comfort to the user but not so wide as to provide discomfort for, among other reasons, the stretching of the vagina. Likewise, the diameter of narrowing 22 should ideally be such that it is most comfortable to the user and may vary from woman to woman from virtually nothing to maybe include being larger than the diameter D of the projection 12. Further, the diameter D of projection 12 on the distal end 16 of projection 12 may be different than the diameter D of projection 12 in the proximal end 18 of projection 12.

In summary, although dimensions of projection 12 (FIG. 30) have been given as typical or representative examples of the length “L” and diameter “D” of projection 12 as well as the location and diameter of narrowing 22, these dimensions are not intended to be limiting. Although specific dimensions have been given here, any dimensions that allow the projection 12 to be in close physical contact, preferably close, comfortable physical contact, with the walls of the user's vagina are intended to be within the scope of the invention. As a result, any length or diameter of projection 12 or location of narrowing 22 that meets the objectives of this invention as spelled out herein are within the scope of the invention.

Base 14 is preferably slightly curved and relatively thin. Base 14 has a contact surface 24, a back surface 26, a bottom end 28 and a top end 30 as well as a base outer surface 32 that is the ultimate “skin” of the base. An outer edge 34 extends around the periphery of base 14. The proximal end 18 of projection 12 attaches to the contact surface 24 of base 14 near the bottom end 28. Base 14 preferably has a width “W” somewhat larger than its thickness “T”. The width W of base 14 is preferably about 1.875 at the bottom end 28, 1.625 inches at the top end 30 and about 1.25 inches midway between the bottom end 28 and the top end 30. The length of base 14 from bottom end 28 to top end 30 is typically about 4.75 inches. The thickness T is typically about 0.25 inches. It is also preferable, although not required, that base 14 have a curvature from the bottom end 28 to the top end 30 in order to conform more closely to the user's anatomy. A typical curvature for base 14 is a radius of about 3.375 inches centered on the distal end 16 of the projection 12.

Contact surface 24 has a ridge 36 running from the projection 12 toward the top end 30. Ridge 36 has a peak 38 that is most displaced from the contact surface 24 of base 14. Ridge 36 has a low height at its peak 38 that tapers to near the outer edge 34. The contact surface 24 with ridge 36 is intended to conform approximately to the shape of the tissue of the vulva between the user's labia. A typical height for peak 38 is about 0.75 inches.

Although dimensions of base 14, including ridge 36 and peak 38, have been given as typical or representative examples (FIGS. 30 and 31), these dimensions are not intended to be limiting. A larger or smaller radius for base 14 than that described above may be desired due to a woman's specific anatomy or a class of women's anatomies in order to produce a comfortable, effective fit for the device 10. Likewise, the exemplary dimensions for the width W and thickness T of base 14 may be varied considerably depending on anatomical considerations or the desire to have more or less mass for base 14. Although specific dimensions have been given here, any dimensions that allow the base 14 to be in close physical contact, preferably close, comfortable physical contact, with the user's vulva are intended to be within the scope of the invention. As a result, any length, width or thickness of base 14 or height of peak 38 or width of ridge 36 that meets the objectives of this invention as spelled out herein are within the scope of the invention.

The device 10 may be constructed so that the projection 12 and base 14 are integrally formed or the projection 12 and base 14 may be made separately and then attached together by means well understood in the art, including but not limited to, adhesives, heat staking, frictional fit, threaded connection, hook and loop connectors, detents and mechanical catches.

As mentioned, projection 12 is connected to base 14. This configuration results in projection 12 protruding from the contact surface 24. Projection 12 preferably protrudes from base 14 at an angle α of between about 45°-90° and most preferably about 73° to approximate the anatomical angle of the user's vagina relative to the external surface area around the vagina. But, it is clear that because women have varying anatomy, the angle α may vary to provide the utmost comfort and effectiveness for an individual woman. So, although an exemplary range for angle α has been given, any angle α that allows the device 10 to come into close physical contact, preferably close, comfortable physical contact, with the user's vagina or vulva or both is within the scope of the invention.

A key function of the device 10 is that it be cool or warm in use to provide maximum benefit and relief to the user. Consequently, device 10 must be made of a material that is capable of being cooled or warmed and remaining cold or warm, respectively, during use. In one embodiment, the material of device 10 has a high heat capacity so that once the device 10 is cooled or warmed prior to use, as will be described hereafter, device 10 will remain cool or warm, respectively, through use of device 10.

In another embodiment of the invention, device 10 is cooled or warmed during use as also will be described hereafter. In this embodiment device 10 is preferably made of a material with a low heat capacity so that the material will readily be cooled or warmed during use. In either of these embodiments, the projection outer surface 20 of the device 10 will come into contact with delicate tissue of the user in and near the user's vagina. As a result, it is also important that at least the projection outer surface 20 be made of material that is non-reactive with the tissue it comes into contact with. Further, it is preferable that the projection outer surface 20 be smooth to facilitate insertion into and contact with the user's vagina.

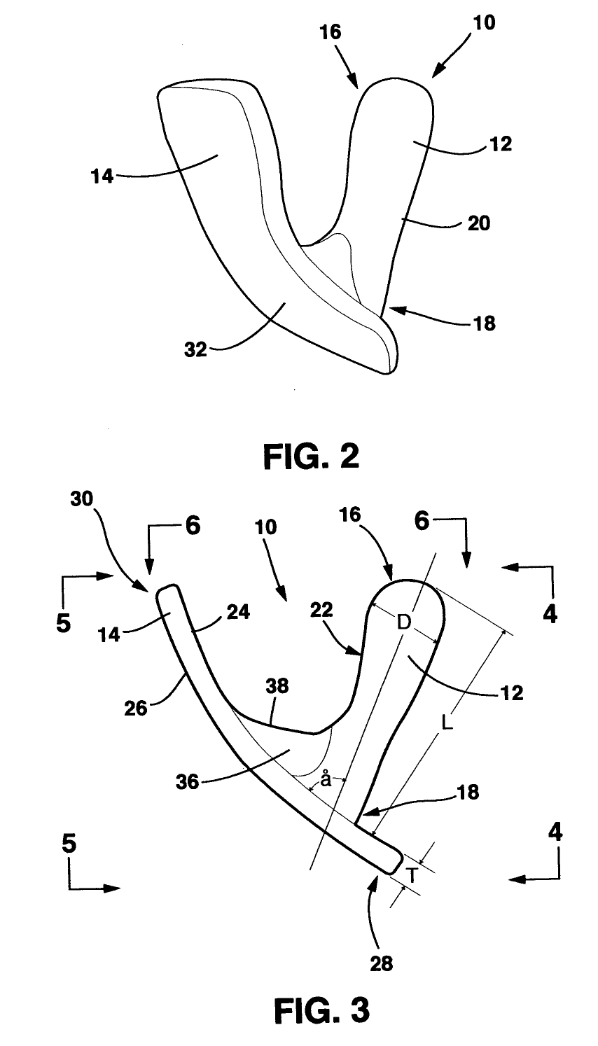

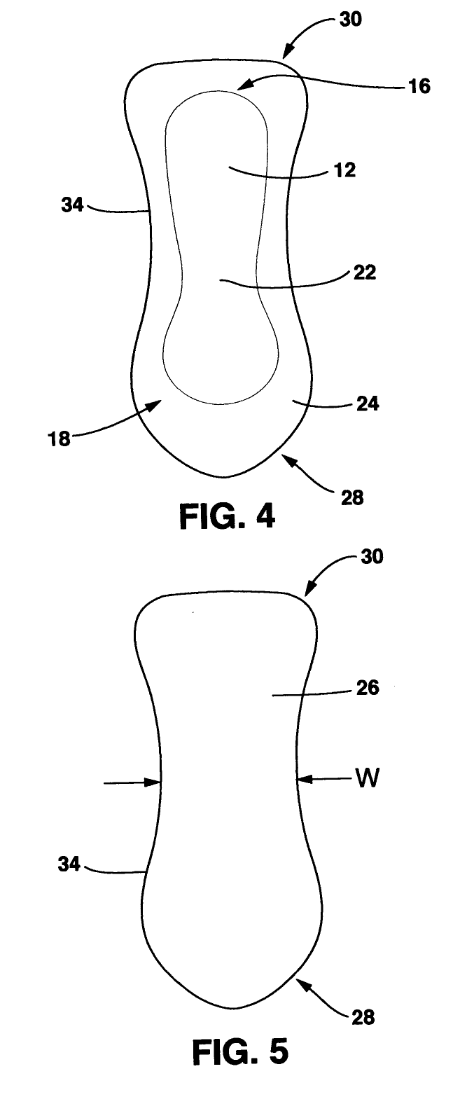

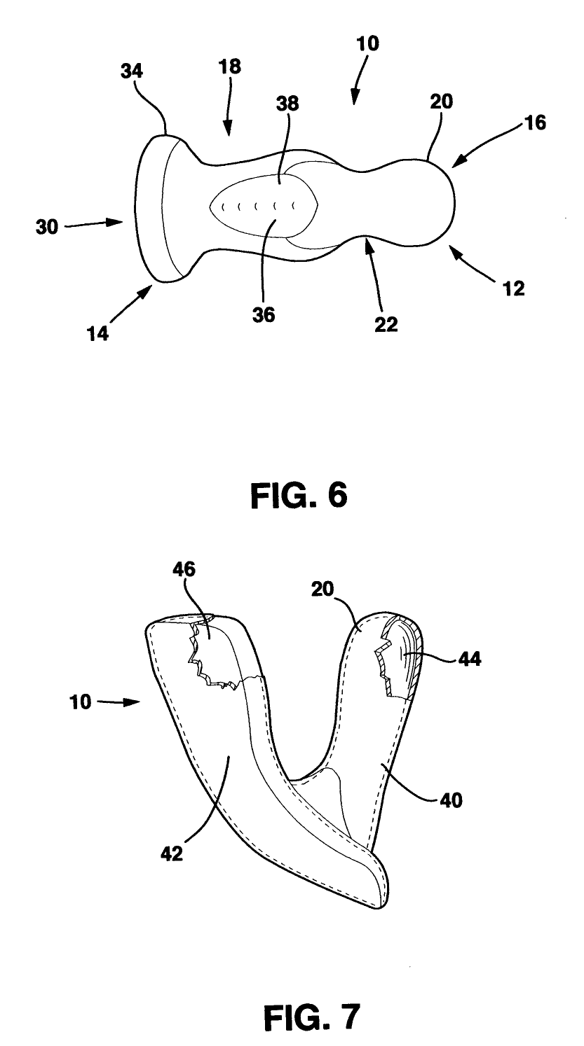

In the embodiment of device 10 shown in FIGS. 2-6, device 10 is made entirely of a material with a high heat capacity, device 10 is preferably made of a hard, smooth plastic having a high heat capacity such as polyvinyl chlorides (PVC) or Metallocene polyethylene (MPE) or ethylene-vinyl acetate (EVA). In this embodiment the device 10 is entirely made of just one material. As stated, the projection 12 and base 14 may be integrally formed by methods such as molding or forming or may be separately made by such methods and joined together as described above.

In a variant of this embodiment shown in FIG. 7, device 10 may have a projection outer surface 20 made of a hard, smooth plastic such as described above forming a projection cavity 40 within the projection outer surface 20. Projection 12 also has a permanent projection inner core 44 entirely filling the projection cavity 40 made of a material having a high heat index. Examples of such material for permanent projection inner core 44 include, but are not limited to, water soluble polymer and those materials set out in the following Table 1 subject to each such material being biocompatible or able to be rendered biocompatible by means such as coating or encapsulation:

TABLE 1

| | Specific |
|---|---|
| | Heat |
| Material | (J/kg° C.) |
| abs resin (high k) | 1506 |
| abs resin (low k) | 1506 |
| acetyl (delrin) | 1464 |
| acrylic (high k) | 1423 |
| acrylic (lucite plexiglass) | 1297 |
| alkyd isocyanate foam | 1674 |
| (density 0.16) | |
| allyl cast resins (high heat | 2343 |
| capacity) | |
| alum | 1548 |
| (k2so4.al2(so4)3.24h2o) | |
| (crystal) | |
| butadiene-acrylonitrile | 1443 |
| rubber + c | |
| butyl rubber | 1966 |
| cellulose acetate (high k) | 1464 |
| cellulose acetate (low k) | 1464 |
| cellulose acetate butyrate | 1464 |
| (high k) | |
| cellulose acetate butyrate | 1464 |
| (low k) | |
| cellulose nitrate | 1464 |
| (pyroxylin) | |
| cellulose proprionate (high | 1464 |
| k) | |
| cellulose proprionate (low | 1464 |
| k) | |
| cellulose triacetate | 1464 |
| chrome alum | 1339 |
| (cr2(so4)3.k2so4.24h2o) | |
| cork ground | 2008 |
| cork ground regranulated | 2008 |
| epsomite (mgso4.7h2o) | 1506 |
| (crystal) | |
| ethyl cellulose (wide range | 2092 |
| of c k) | |
| ethyl vinyl acetate | 2301 |
| fluid heat sink (d = 1 | 4.18E+12 |
| c = k = 10**9) | |
| fluid well stirred (d = c = 1 | 4184 |
| k = 10**9) | |
| melamine (high dens high | 1674 |
| k) | |
| melamine (low dens low | 1674 |
| k) | |
| melamine alpha cellulose | 1674 |
| filled | |
| melamine asbestos filled | 1674 |
| (mst 95-205) | |
| melamine cellulose filled | 1674 |
| (mst 95-205) | |
| melamine fabric or flock | 1674 |
| filled | |
| melamine glass fiber filled | 1674 |
| (mst 205) | |
| methyl methacrylate | 1464 |
| neoprene rubber | 2176 |
| nitrile rubber | 1966 |
| nitrocellulose h.e. (12.7 n) | 1255 |
| nylon 6 11 66 610 | 1527 |
| (polycaprolactam) | |
| nylon glass filled | 1339 |
| petn h.e. | 1674 |
| phenolic resin pressed | 1255 |
| types 40 50 | |
| phenolic cast asbestos | 1255 |
| filler | |
| phenolic cast no filler | 1464 |
| polystyrene general | 1423 |
| purpose | |
| polystyrene modified | 1339 |
| polystyrene prefoamed | 1130 |
| rigid dow q103 | |
| Polysulfone | 1255 |
| polyurethane foam flexible | 1757 |
| polyurethane foamed-in- | 1674 |
| place rigid | |
| polystyrene general | 1423 |
| purpose | |
| polyurethane rubber 1-100 | 1674 |
| polyvinyl alcohol | 1255 |
| polyvinyl butyral | 1674 |
| polyvinyl carbazole | 1297 |
| polyvinyl chloride acytate | 1674 |
| flexible | |
| polyvinyl chloride flexible | 1674 |
| polyvinyl tpx-r | 2176 |
| polyvinylidene chloride | 1339 |
| polyvinylidene chloride | 1339 |
| film | |
| polyvinylidene fluoride | 1381 |
| (kynar) | |
| porcelain 4811 (beo) | 1004 |
| potassium alum | 1548 |
| (k2so4.al2(so4)3.24h2o) | |
| potassium aluminum | 1255 |
| silicate (orthoclas) | |
| potassium ferrocyanide | 1130 |
| (k4fe(cn)6.3h2o) | |
| quartz flour fine (h2o 21 | 1464 |
| pc) | |
| quartz powder coarse (h2o | 1590 |
| 24) | |
| rubber buna with carbon | 1757 |
| black | |
| rubber butyl | 1966 |
| rubber dielectric mix | 2092 |
| rubber high k | 2092 |
| rubber neoprene | 2176 |
| rubber nitrile | 1966 |
| rubber polyurethane | 1674 |
| elastomer 1-100 | |
| silicone foam flexible (lrl) | 1339 |
| silicone foam rigid | 1339 |
| various | |
| silicone rubber high k (see | 1255 |
| ref 5) | |
| silicone rubber low k (see | 1255 |
| ref 5) | |
| silicone rubber medium k | 1255 |
| (see ref 5) | |
| silicone rubber rtv 521 | 1255 |
| and 093-009 | |
| silicone molded various | 1046 |
| fillers | |
| Sodium | 1213 |
| sodium aluminum silicate | 1339 |
| hydrate (xtal) | |
| sodium fluoride (naf) | 1113 |
| (prsd 8 kb) | |
| soil - sandy, clay, quartz, | 1004-2929 |
| loam | |
| Teflon | 1004 |
| tetryl h.e. | 1255 |
| tnt h.e. (246- | 1372 |
| trinitrotoluene) (cast) | |
| topaz (2alfo.sio2) (a-axis) | 1255 |
| topaz (2alfo.sio2) (c-axis) | 1255 |
| water (h2o) (liquid) | 4184 |
| water (h2o) (solid) (ice) | 4217 |
| Water (h2o) snow | 2092 |
| Wood - balsa, cypress, | 1255-2385 |
| mahogany, maple, pine, | |
| oak, spruce, fir, teak | |

Also as shown in FIG. 7, in this embodiment base 14 has a base outer surface 32 made of a hard, smooth plastic such as described above forming a base cavity 42 within the base outer surface 32. Base 14 also has a permanent base inner core 46 entirely filling the base cavity 42 made of a material having a high heat index such as water soluble polymers or those shown in Table 1.

Although the embodiment shown in FIG. 7 and described above shows both projection 12 and base 14 having permanent inner cores, 44, 46, respectively, it is also within the scope of the invention for either projection 12 or base 14 to have a respective inner core 44, 46 without the other having an inner core 44, 46. It is also within the scope of the invention for either projection 12 or base 14 to be made of a single solid material irrespective of whether the other has an inner core 44, 46, respectively.

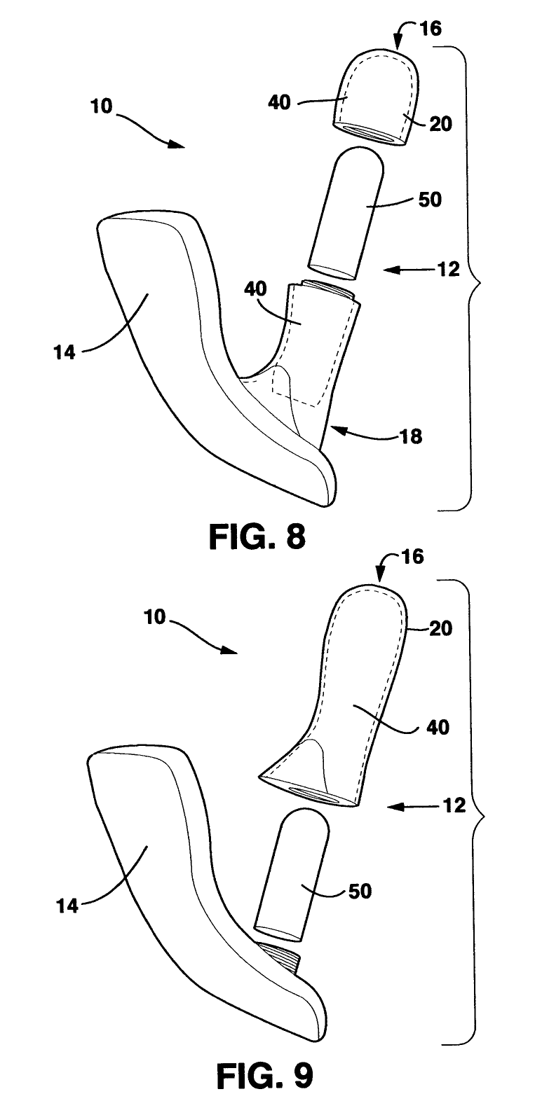

In a further variant of this embodiment shown in FIGS. 8-11, device 10 has a projection outer surface 20 made of a hard, smooth plastic such as described above but also has a projection cavity 40 within projection outer surface 20. Projection cavity 40 holds a removable projection inner core 48 made of a material having a high heat index such as water soluble polymers or those shown in Table 1 or may be adapted to hold ice, cold water or a combination of ice and water.

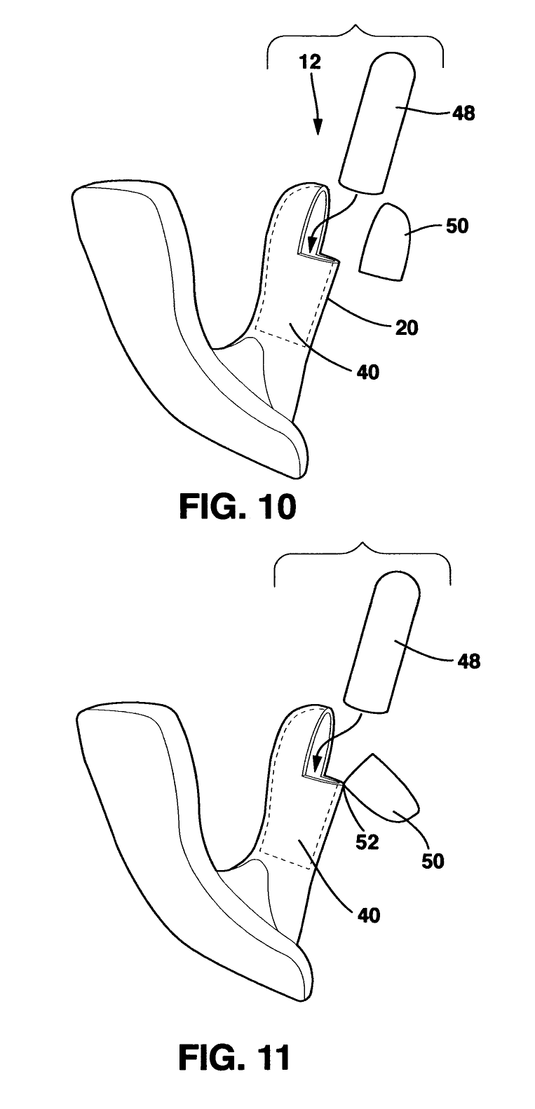

Access to projection cavity 40 is provided by removing a portion of projection outer surface 20 (FIGS. 8-9) or through a projection access door 50 to projection cavity 40 (FIGS. 10-11). In the embodiment shown in FIG. 8, the distal end 16 of projection 12 is removed thereby exposing the projection cavity 40. Distal end 16 may be removably attached to projection 12 by means well understood in the art including, but not limited to, screw threads, friction fit, mechanical connection, detents, snaps, catches, hook and loop connectors, etc.

Alternately, as shown in FIG. 9, a substantial portion of projection 12 may be separated from the remaining portion of projection 12 near the proximal end of projection 12 and the two portions of projection 12 connected to each other as described above in connection with the embodiment shown in FIG. 8. It is also clear that projection 12 may be separated into parts at virtually any point to provide access to the inner projection cavity 40 where the separate parts of projection 12 may be attached to each other as described above.

Where a projection access door 50 is provided to provide access to inner projection cavity 40, as shown in FIG. 10, a portion of projection outer surface 20 may be the removable to create projection access door 50. In one variant of this embodiment, shown in FIG. 10, projection access door 50 is removably attached to the rest of projection outer surface 20 through means well understood in the art including, but not limited to, a frictional or mechanical fit, snaps, catches, hook and loop connectors, detents, etc. In a variant of this embodiment, shown in FIG. 11, projection access door 50 is be attached to the rest of projection outer surface 20 by a hinge 52 so that projection access door 50 pivots away from the rest of the projection outer surface 20 to provide access to the inner projection cavity 40.

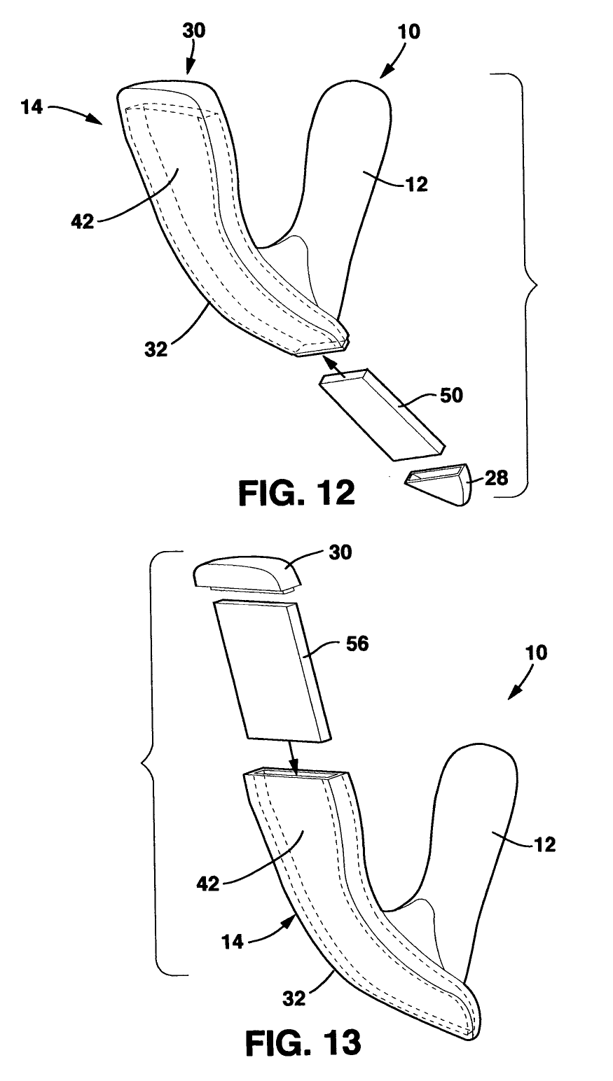

In an additional variant of this embodiment shown in FIGS. 12-16, device 10 has a base outer surface 32 made of a hard, smooth plastic such as described above but also has a base cavity 42 within base outer surface 32. Base cavity 42 holds a removable base inner core 54 made of a material having a high heat index such as water soluble polymers or those shown in Table 1 or may be adapted to hold ice, cold water or a combination of ice and water.

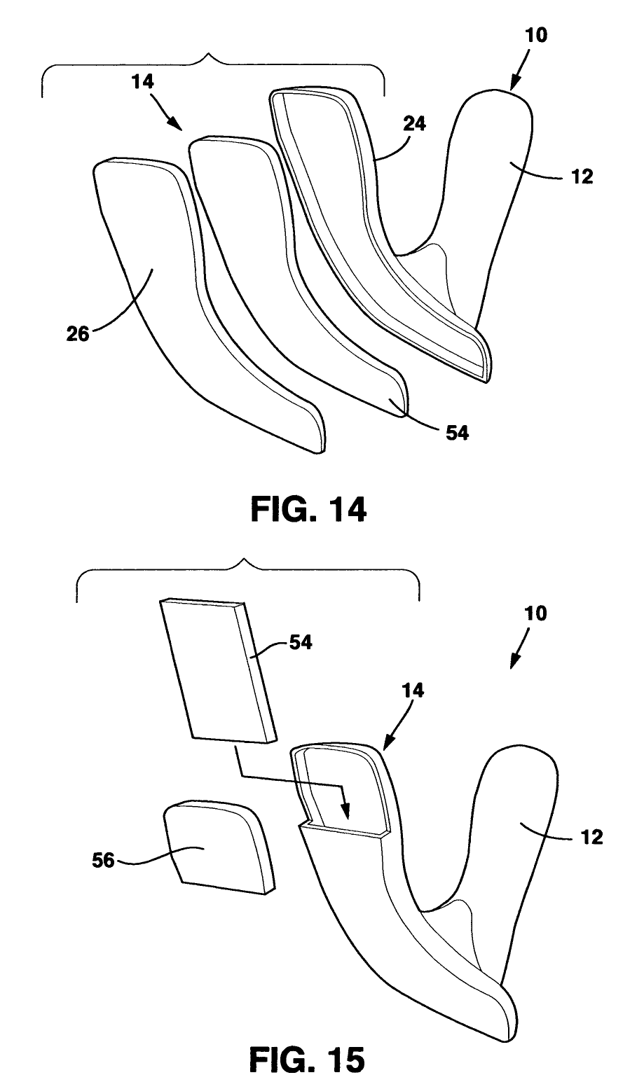

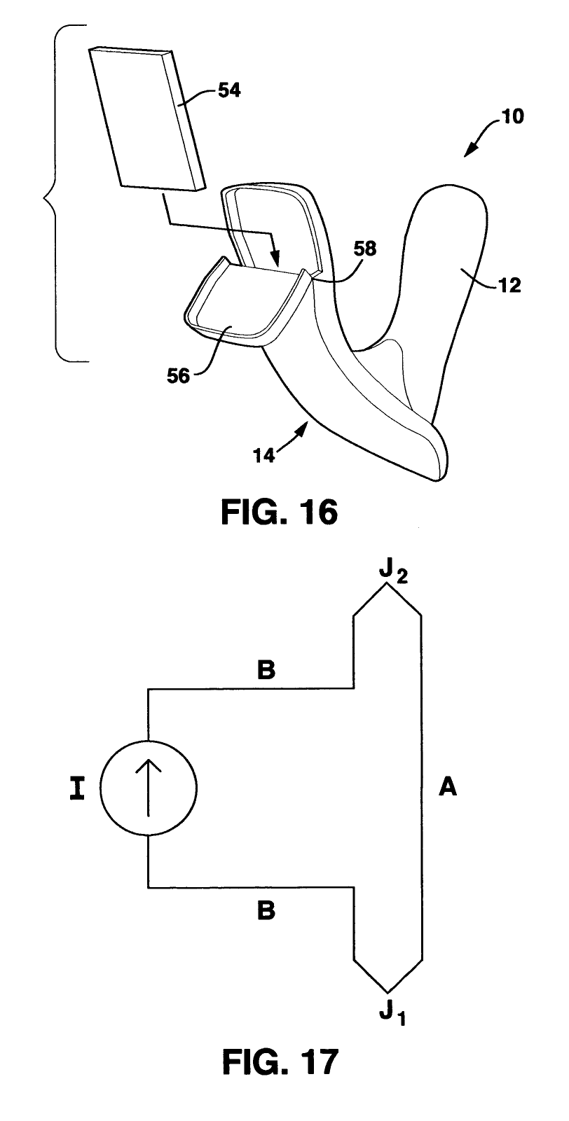

Access to base cavity 42 is provided by removing a portion of base outer surface 32 through removal of either the bottom end 28 (FIG. 12) or top end 30 (FIG. 13) of base 14, the separation of contact surface 26 from the back surface 24 (FIG. 14) or through a base access door 56 to base cavity 42 (FIGS. 15-16). Where a portion of base 14 is removed, thereby exposing the base cavity 42, either the bottom end 28 (FIG. 12) or top end 30 (FIG. 13) may be removably attached to base 14 so that removal of either exposes the base cavity 42. Either bottom end 28 or top end 30 may be removably attached to base 14 by means well understood in the art including, but not limited to, screw threads, friction fit, mechanical fit, snaps, catches, hook and loop connectors, detents, etc.

Alternately, the contact surface 24 of base 14 may be separated from the back surface 24 of base 14 along outer edge 34 (FIG. 14). In this embodiment, contact surface 24 and back surface 26 may be attached by means well understood in the art including, but not limited to, screw threads, friction fit, mechanical fit, snaps, catches, hook and loop connectors, detents, etc.

Where a base access door 56 is provided to inner base cavity 42, a portion of base outer surface 32 may be the removable to create base access door 56 (FIGS. 15-16). In this embodiment, base access door 56 is removably attached to the rest of base outer surface 32 through means well understood in the art including, but not limited to, a frictional or mechanical fit, snaps, catches, hook and loop connectors, detents, etc. (FIG. 15). In a variant of this embodiment, shown in FIG. 16, base access door 56 is be attached to the rest of base outer surface 32 by a hinge 58 so that base access door 56 pivots away from the rest of the base outer surface 32 to provide access to the inner base cavity 42.

As mentioned, in the embodiments having a removable projection inner core 48 or removable base inner core 54, the removable projection inner core 48 is placed entirely within projection cavity 40 filling projection cavity 40 and the removable base inner core 54 is placed entirely within base cavity 42 filling base cavity 42, respectively. In these embodiments, the removable projection inner core 48 or removable base inner core 54 may be separated from projection cavity 40 and base cavity 42, respectively, cooled or warmed and then replaced in projection cavity 40 and base cavity 42, respectively, to cool or warm, respectively, the projection outer surface 20 and base outer surface 32, respectively. Alternately, the removable projection inner core 48 or removable base inner core 54 may be retained in projection cavity 40 and base cavity 42, respectively, and cooled or warmed as part of the cooling or warming, respectively, of the device 10 generally.

It is clear that the embodiments of device 10 containing a removable projection inner core 48 and its corresponding projection cavity 40 and removable base inner core 54 and its corresponding base cavity 42 can be combined into a single device 10 having both a removable projection inner core 48 and its corresponding projection cavity 40 and removable base inner core 54 and its corresponding base cavity 42.

As stated, a key function of the device 10 is that it be cool or warm in use to provide maximum benefit and relief to the user. One way to accomplish this cooling or warming is to place device 10 in an environment that is cold or warm and then letting the interaction between the device 10 and the cold or warm environment cool or warm the device 10. One such cooling environment is a refrigerator or freezer. Most homes have both a refrigerator and either an integral or separate freezer. In this embodiment, the device 10, in whatever form it takes, is intended to be placed in either the refrigerator, freezer or other cold environment and thereby cooled. One such warming environment is an oven or warm water bath or in a device to heat the device 10 or portions thereof such as a microwave oven. Most homes have an oven or a microwave oven or have warm water. In this embodiment, the device 10, in whatever form it takes, is intended to be placed in either a conventional or microwave oven or in a warm water bath or other warm environment and thereby warmed.

Device 10 may also be cooled or warmed sufficiently to be therapeutic and comforting to the user by means other than placing device 10 in a cold or warm environment until device 10 is cooled or warmed. Another way this cooling or warming can be accomplished is through the use of a Peltier module, sometimes called a heat pump or, in a cooling mode, an active Peltier refrigerator or Peltier cooler or in a warming mode a Peltier heater. A Peltier module includes a Peltier circuit (FIG. 17).

A Peltier circuit uses the Peltier effect. The Peltier effect is the creation or removal of heat occurring when an electric current from a power source I is passed through two dissimilar metals or semiconductors A, B connected together at two junctions J₁, J₂. The electric current drives a transfer of heat from one junction to the other. This causes one junction to heat up while the other junction cools off. Heat sinks are often placed in association with each junction. These heat sinks become cold or hot, respectively. Peltier circuits are quite compact, reliable and efficient.

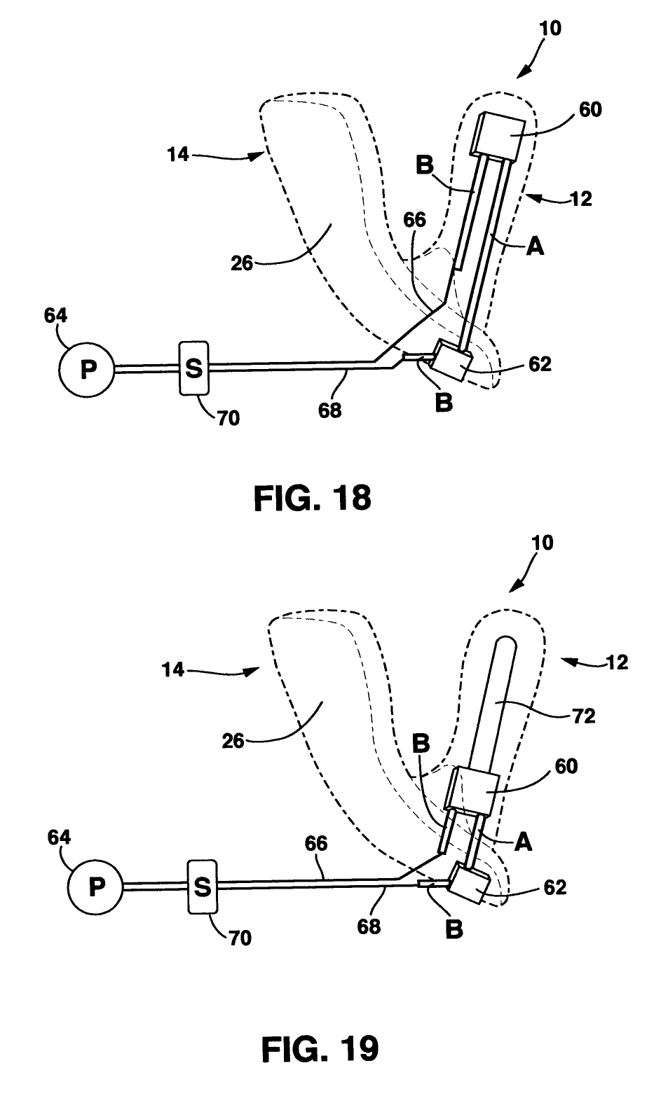

FIG. 18 shows an embodiment of the invention incorporating a Peltier module. In this embodiment, the device 10 contains a head unit 60 comprising one junction of a Peltier circuit and an end unit 62 comprising the other junction of a Peltier circuit. A power source 64 provides power for the Peltier circuit and is connected to head unit 60 by a first connection wire 66 and to end unit 62 by a second connection wire 68. A switch 70 completes the electrical circuit and controls the operation of the Peltier circuit.

In the embodiment shown in FIG. 18, the head unit 60 is preferably located within and in functional contact with the projection 12. Alternately, as shown in FIG. 19, the head unit 60 is located on the contact surface 24 of base 14 and has a projection heat sink 72 in contact with head unit 60 extending into projection 12. In either of these ways, as the head unit 60 becomes cooled through the operation of the Peltier circuit, the projection 12 becomes cooled. End unit 62 is preferably located away from the user's genitals, preferably on the back surface 26 of the base 14. Alternately, the junctions of this embodiment could be reversed so that the first junction in head unit 60 heats instead of cools and the end unit 62 cools instead of heats.

Power source 64 may be either a battery or a power supply that receives power from common household electrical current Switch 70 is preferably located conveniently for the user such as on the back surface 26 of base 14 or on either the first or second connection wires 66, 68.

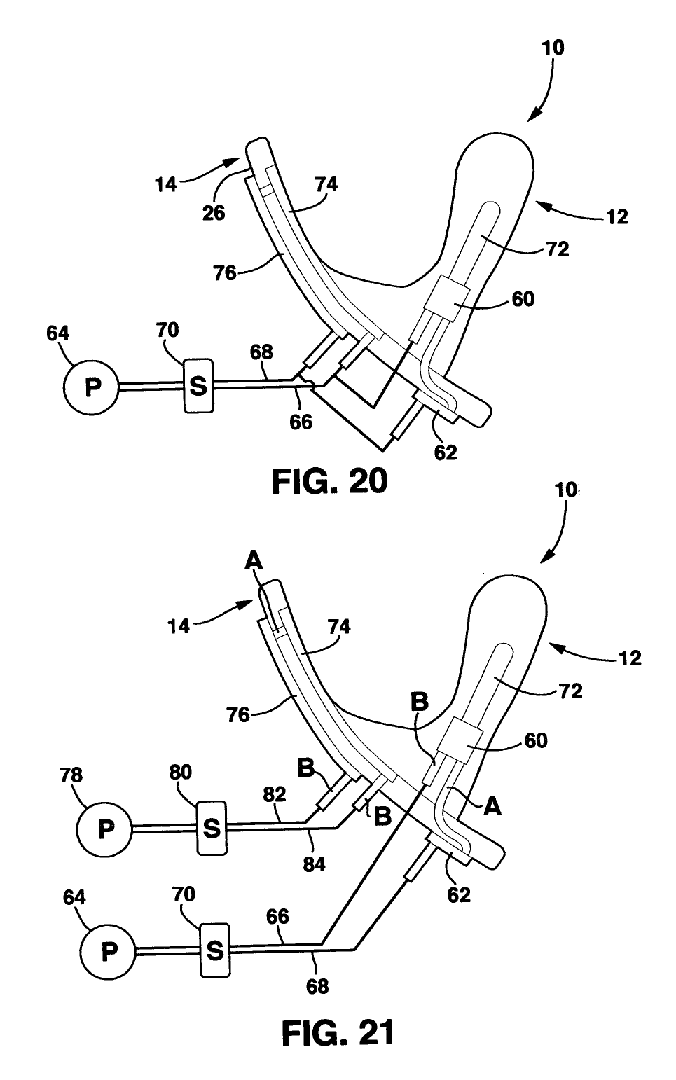

In a variant of this embodiment shown schematically in FIG. 20 a separate base head unit 74 is placed in contact with the base 14 so that cooling or warming of the base head unit 74 cools or warms, respectively, the base 14. In this embodiment, base head unit 74 has a corresponding base end unit 76, preferably located on back surface 26 of the base 14. Base head unit 74 and base end unit 76 may part of the Peltier circuit that powers the head unit 60 in that both head unit 60 and base heat unit 74 and end unit 62 and base end unit 76 share than the same power source 64 and switch 70. Alternately, as shown schematically in FIG. 21, base head unit 74 and base end unit 76 has its own power source 78 and switch 80 similar or identical to power source 64 and switch 70 with the necessary connection wires 82, 84. In either of these two embodiments, the base head unit 74 and base end unit 76 may be used with or without the head unit 60 and end unit 62.

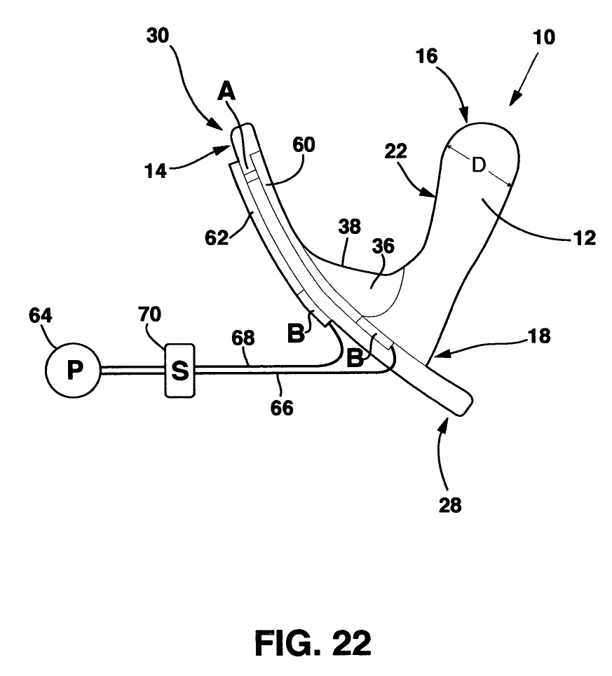

Finally, in a further embodiment shown in FIG. 22, the head unit 60 is preferably located within and in functional contact with the base 14. In this way, as the head unit 60 becomes cooled or warmed through the operation of the Peltier circuit, the base 14 becomes cooled or warmed, respectively. End unit 62 is preferably located away from the user's genitals, preferably on the back surface 26 of the base 14.

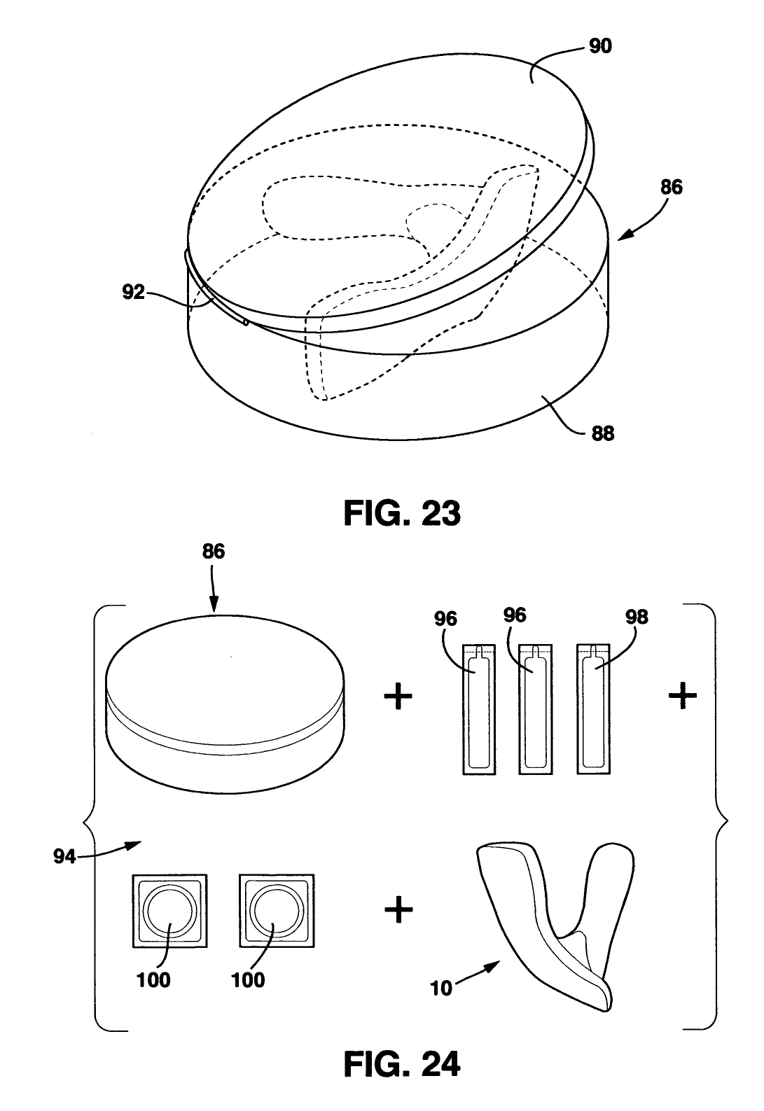

Because of the personal nature of the use of the device 10, in one embodiment of the invention, shown in FIG. 23, the device 10 also includes a discreet container 86 for containing the device 10. Container 86 has a boxlike or storage configuration that not only contains and protects the device 10 but also provides a holder for the device 10 while the device 10 is in the refrigerator or freezer getting cold. Container 86 may come in a box configuration with a body 88 and a pivoting lid 90 attached to body 88 by a hinge 92 (FIG. 23). Alternately, container 86 may again have a body 88 and a lid 90 that are removably connected through means such as frictional contact, mechanical connection, detents, catches, hook and loop connectors, etc. It is clear that an almost endless array of configurations for container 86 will occur to those skilled in the art of containers. Container 86 is most preferably made of a material with a high heat capacity such as a hard, smooth plastic with a high heat capacity like polyvinyl chlorides (PVC) or Metallocene polyethylene (MPE) or ethylene-vinyl acetate (EVA) to facilitate the cooling or warming of device 10 but may also be made of other plastics, ceramics, paper or metal as also will be clear to those skilled in the art of containers. In the embodiment having a removable projection inner core 48 or removable base inner core 54, either the container 86 used to store device 10 or a separate container may be used to cool or warm removable projection inner core 48 or removable base inner core 54 by placing this container with removable projection inner core 48 or removable base inner core 54 in a refrigerator, freezer, conventional or microwave oven or warm water bath to produce the desired cooling or warming, respectively.

Another embodiment of the device 10 shown in FIG. 24 involves creating a kit 94 that includes the device 10 and therapeutic jellies 96. In addition, the kit 94 may include the container 86. These therapeutic jellies 96 may include a lubricant 98 to assist the user in inserting the device 10. The therapeutic jellies 96 also may include therapeutic agents such as anti-inflammatory, antibiotic, anti-fungal or moisturizing ingredients such as are well known to medical and holistic science for specific relief for vaginitis or other conditions affecting a woman's genitals and vagina particularly due to inflammation of the vaginal and vulvar tissue resulting from trauma or irritation. The lubricant 98 may include, but is not limited to, lubricating gel such as K-Y Jelly® sold by the Johnson & Johnson Company of New Brunswick, N.J. The purpose of therapeutic jellies 96 and lubricant 98 is to facilitate the insertion of the projection 12 into the vagina, to perform a therapeutic function related to treating the malady causing the irritation or to provide a topical relief to the user.

The kit 94 may also include one or more condoms 100 for assuring sterility of the device 10. The use of a condom 100 with the device 10 may be especially appropriate for post-partum use because the cervix may still dilated and therefore susceptible to the introduction of bacteria or other unwanted agents by the projection 12.

In one embodiment of the device 10, the device 10 is intended to be reusable. In this embodiment, because of the material of which device 10 is made, device 10 is intended to be cleansed with soap and hot water or with an approved disinfectant like hydrogen peroxide. Thereafter, the device 10 should be dried and readied for cooling or warming preparatory to its next use.

In another embodiment, device 10 is disposable. In this embodiment, after each use of device 10 the device 10 is discarded. However, in this embodiment, the container 86 used to contain device 10 may be reused or may also be disposed of with device 10.

In use where the device 10 is cooled prior to use, device 10, of course, must be cooled prior to use. One way of accomplishing this cooling is by putting device 10 in a cool or cold environment such as a refrigerator or freezer, either with or without a container 86, and letting the device 10 cool to the desired temperature. Where the freezer has a temperature lower that that desired for ultimate use of the device 10, the freezer may be used to cool the device to a desired temperature and then removed from the freezer before the device 10 cools to a temperature lower than desired.

Another way of cooling device 10 is to cool the removable projection inner core 48, removable base inner core 54 or both by placing them, or them with a container 86, in a refrigerator or freezer until they are at the desired temperature. Prior to use, removable projection inner core 48, removable base inner core 54 or both, in their cool or cold condition, are placed in projection cavity 40 or base cavity 42, respectively.

A further way of cooling device 10 is to place ice, cold water or both into the respective projection cavity 40 or base cavity 42 prior to use. The objective of these methods of cooling device 10 prior to use is to get device 10, particularly the projection outer surface 20 and base outer surface 32 to the desired temperature to produce optimum relief. It is believed that this temperature range extends from about 15° F. to about 50° F. to with a temperature of about 25° F. believed to be most preferred.

An additional way of cooling device 10 is to activate the Peltier circuit so that cooling will begin. When using the embodiment of device 10 with a Peltier circuit, it is desirable to activate the Peltier circuit by activating the switch 70 or switch 80 so that the device 10 can be cooled to the desired temperature prior to use.

In use where the device 10 is warmed prior to use, device 10, of course, must then be warmed prior to use. One way of accomplishing this warming is by putting device 10 in a warm or hot environment such as a an oven or warm water bath or in a device to heat the device 10 or portions thereof such as a microwave oven, either with or without a container 86, and letting the device 10 warm to the desired temperature. Where the oven or warm water bath has a temperature higher that that desired for ultimate use of the device 10, the oven or warm water bath may be used to heat the device to a desired temperature and then removed from the oven or bath before the device 10 warms to a temperature higher than desired.

Another way of warming device 10 is to warm the removable projection inner core 48, removable base inner core 54 or both by placing them, or them with a container 86, in an oven or warm water bath until they are at the desired temperature. Prior to use, removable projection inner core 48, removable base inner core 54 or both, in their warm condition, are placed in projection cavity 40 or base cavity 42, respectively.

A further way of warming device 10 is to place warm water or other warm liquid into the respective projection cavity 40 or base cavity 42 prior to use. The objective of these methods of warming device 10 prior to use is to get device 10, particularly the projection outer surface 20 and base outer surface 32 to the desired temperature to produce optimum relief. It is believed that this temperature range extends from about 97° F. (low end of normal human core body temperature) to about 110° F. (upper end of normal topical heat toleration) with a temperature between about 100°-105° F. believed to be preferred and a temperature of about 103° F. believed to be most preferred.

An additional way of warming device 10 is to activate the Peltier circuit so that warming will begin. When using the embodiment of device 10 with a Peltier circuit, it is desirable to activate the Peltier circuit by activating the switch 70 or switch 80 so that the device 10 can be warmed to the desired temperature prior to use.

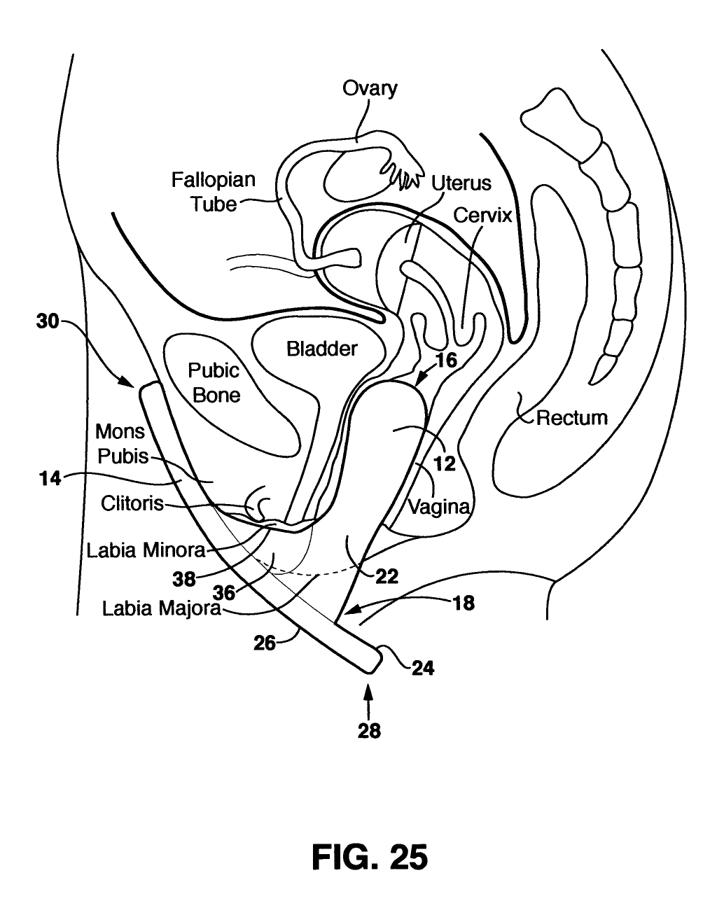

Once the device 10 is cooled or warmed to the desired temperature, the projection 12 is inserted into the user's vagina (FIG. 25). It may be desirable to coat the projection 12 with a therapeutic jelly 96 or lubricant 98 to lubricate projection 12 to facilitate insertion or to provide a topical, therapeutic agent to the vagina. It may also be desired to place a therapeutic jelly 96 on the contact surface 24 of the base 14 to provide a topical, therapeutic agent to the user's vulva.

As the projection 12 is moved farther into the user's vagina, the ridge 36 will come into contact with the user's vulva between the labia. The peak shape of ridge 36 conforms with the user's anatomy to bring the ridge 36 and the contact surface 24 into conformal contact with the user. Because the projection 12 and base 14 are cool or warm, the therapeutic benefits of this cool or warm contact will be felt by the user both within the vagina and in the vulva area.

If the device 10 eventually ceases to be cool or warm enough to provide the desired therapeutic benefit or comfort, the user may remove the device 10. Where the device 10 is reusable, the device 10 may be recooled or rewarmed and thereafter reused. Where the device 10 is disposable and the user desires to continue treatment, the user should cool or warm another device 10 as described above and use the device as described above. Where the device 10 uses a Peltier circuit, the Peltier circuit can continue to provide cooling or warming while in place in contact with the user's genitals.

Particularly where the device 10 is cooled to a temperature below freezing, it may be desirable to put a protective cover over at least a portion of the device 10 to prevent the device 10 and moist tissue of the user from sticking. This protective cover could be a condom 100 or a condom-like sheath made of paper, cardboard or cloth. It may also be desirable to provide the protective cover described above when the device 10 is used as a warming device.

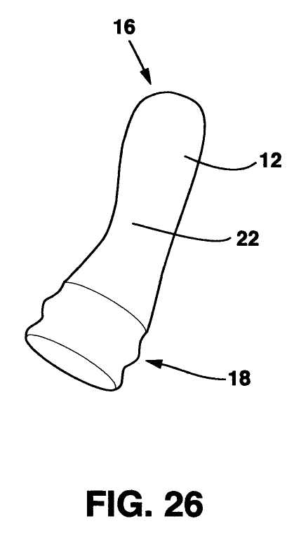

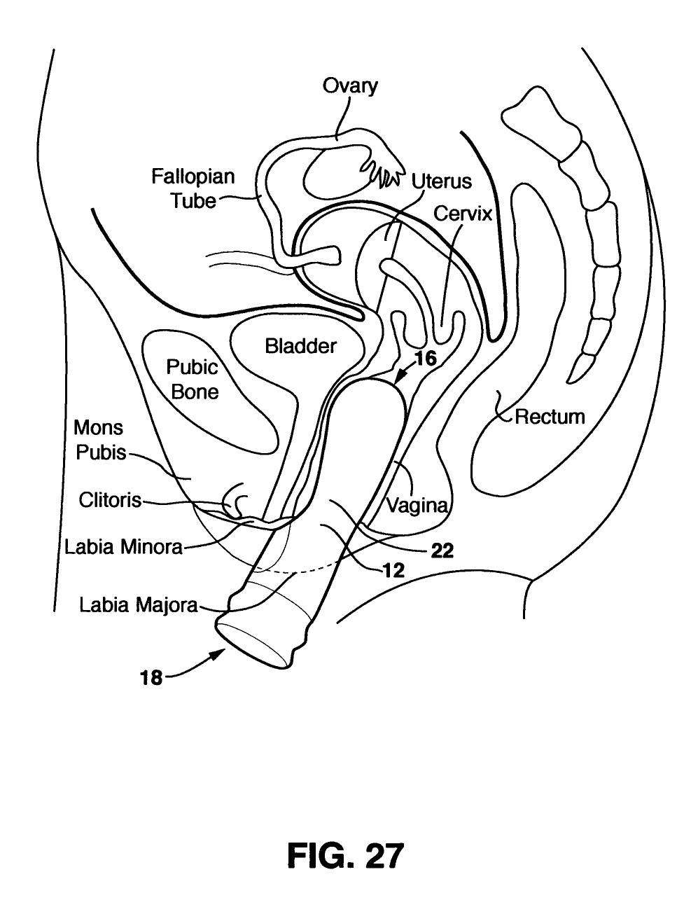

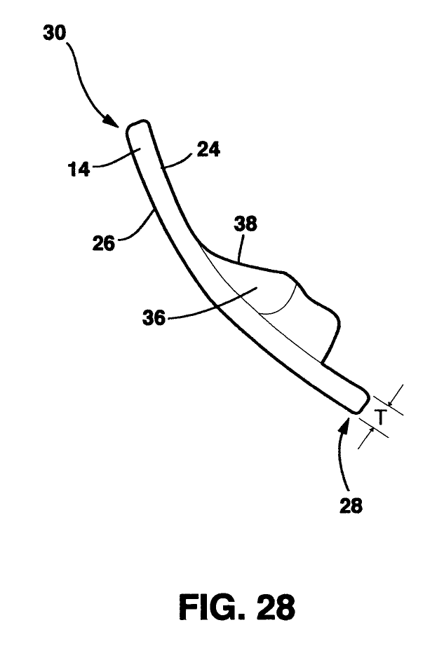

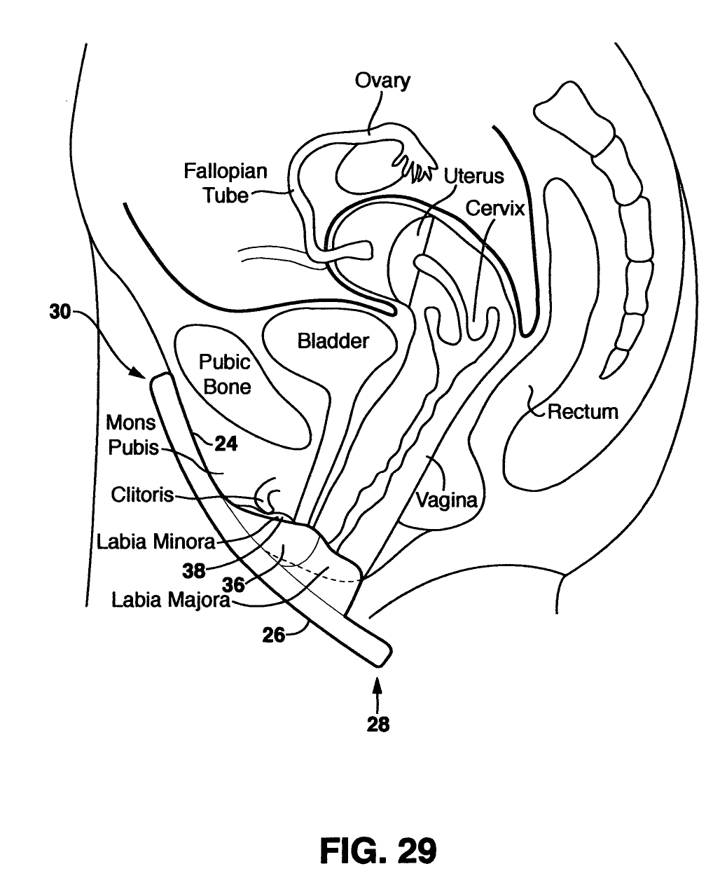

Although the device 10 has been described as having both a projection 12 and a base 14, it is also within the scope of the invention for the device 10 to have only a projection 12 (FIG. 26) or only a base 14 (FIG. 28), each in all the various embodiments and variations described above. In these embodiments, the device 10 is brought into contact with the female genitals. With respect to the embodiment having only a projection 12, the projection 12 in use is inserted into the user's vagina (FIG. 27). With respect to the embodiment having only a base 14, the base 14 is brought into contact with the user's vulva (FIG. 29).

The invention heretofore has been described primarily in connection with embodiments directed to human use. However, the underlying principle of the device applies to all females and vaginitis associated with their genitals and specifically with their vulvas. Consequently, the invention described herein could be adapted for use in animals as well, both for treating vaginitis and other vulvar related inflammation, irritation and itching, but also to warm the vulva and vagina to aid in or as an inducement for mating. It is clear that because of the wide range in sizes and anatomies associated with the various female animals the present invention could be applied to that the specific dimensions of the device 10 will have to be adjusted, according to the principles set out in the description herein, to the animals who will use the device 10.

The invention has been described herein in connection with specific embodiments and dimensions. It is to be understood, however, that the description given herein has been given for the purpose of explaining and illustrating the invention and are not intended to limit the scope of the invention. It is to be further understood that changes and modifications to the descriptions given herein will occur to those skilled in the art. Therefore, the scope of the invention should be limited only by the scope of the following claims.

What is claimed is:

1. A vaginal or vulvar cooling or warming device comprising:
a base;
an elongated projection having a distal end and a proximal end and a projection outer surface, the projection attached to base at the proximal end.

2. The cooling or warming device of claim 1 wherein the projection is hourglass shaped so that the diameter of the projection between the distal end and the proximal end is somewhat less than the diameters of either the distal end or the proximal end.

3. The cooling or warming device of claim 1 wherein the diameter of the projection between the distal end and the proximal end is larger than the diameter of either the projection at its distal end or at its proximal end.

4. The cooling or warming device of claim 1 wherein the projection has a constant diameter between the distal end and the proximal end.

5. The cooling or warming device of claim 1 wherein the projection has an increasing diameter between the distal end and the proximal end.

6. The cooling or warming device of claim 1 wherein the projection has a decreasing diameter between the distal end and the proximal end.

7. The cooling or warming device of claim 1 wherein the distal end of the projection is rounded to facilitate its insertion into a user's vagina.

8. The cooling or warming device of claim 1 wherein the length of the projection is between about 0 inches and about 4.7 inches.

9. The cooling or warming device of claim 1 wherein the diameter of the projection is between about slightly larger than 0 inches and about 1.25 inches.

10. The cooling or warming device of claim 1 wherein the base is slightly curved and relatively thin.

11. The cooling or warming device of claim 10 wherein the base has a width somewhat larger than its thickness.

12. The cooling or warming device of claim 1 wherein the base has a contact surface, a back surface, a bottom end, a top end, a base outer surface and an outer edge that extends around the periphery of the base.

13. The cooling or warming device of claim 12 wherein the contact surface has a ridge running from the projection toward the top end.

14. The cooling or warming device of claim 13 wherein the ridge has a peak that is displaced from the contact surface of the base.

15. The cooling or warming device of claim 14 wherein the ridge has a low height at its peak that tapers to near the outer edge.

16. The cooling or warming device of claim 13 wherein the contact surface and ridge is adapted to conform approximately to the shape of the tissue of the vulva between a user's labia.

17. The cooling or warming device of claim 1 wherein the projection and the base are integrally formed.

18. The cooling or warming device of claim 1 wherein the projection and the base are made separately and then attached together.

19. The cooling or warming device of claim 1 wherein the projection protrudes from the contact surface at an angle of between about 45° to 90° approximating the anatomical angle of a user's vagina relative to the vulva.

20. The cooling or warming device of claim 1 wherein the cooling or warming device is made substantially of a material of device having a high heat capacity whereby once the cooling or warming device is cooled or warmed prior to use, the cooling or warming device will remain cool or warm, respectively, through use of the cooling or warming device.

21. The cooling or warming device of claim 1 wherein the cooling or warming device is made substantially of a material with a low heat capacity whereby the material of the cooling or warming device will readily be cooled or warmed during use.

22. The cooling or warming device of claim 21 wherein the cooling or warming device is made substantially of a material chosen from a group consisting of polyvinyl chlorides (PVC) or Metallocene polyethylene (MPE) or ethylene-vinyl acetate (EVA).

23. The cooling or warming device of claim 21 wherein the cooling or warming device is made entirely of one material.

24. The cooling or warming device of claim 1 wherein at least the projection outer surface is made of material that is non-reactive with a user's tissue that the cooling or warming device comes into contact with.

25. The cooling or warming device of claim 1 wherein at least the projection outer surface be smooth to facilitate insertion into and contact with a user's vagina.

26. The cooling or warming device of claim 1 wherein the projection has:
a projection outer surface made of a hard, smooth plastic forming a projection cavity within the projection; and
a permanent inner core filling the projection cavity, the permanent inner core made of a material having a high heat index.

27. The cooling or warming device of claim 26 wherein the permanent inner core is made of a material chosen from a group consisting of polyvinyl chlorides (PVC) or Metallocene polyethylene (MPE) or ethylene-vinyl acetate (EVA).

28. The cooling or warming device of claim 1 wherein the projection has:
a projection outer surface made of a hard, smooth plastic forming a projection cavity within the projection; and
a removable inner core filling the projection cavity, the removable inner core made of a material having a high heat index.

29. The cooling or warming device of claim 28 wherein the removable inner core is made of a material chosen from a group consisting of polyvinyl chlorides (PVC) or Metallocene polyethylene (MPE) or ethylene-vinyl acetate (EVA).

30. The cooling or warming device of claim 1 wherein the projection has a projection outer surface made of a hard, smooth plastic forming a projection cavity within the projection.

31. The cooling or warming device of claim 30 wherein access to the projection cavity is provided by removing a portion of the projection outer surface.

32. The cooling or warming device of claim 31 wherein the distal end of the projection is removed thereby exposing the projection cavity.

33. The cooling or warming device of claim 32 wherein the distal end of the projection is attached to the remainder of the projection by attachment means chosen from the group consisting of screw threads, friction fit, hook and loop connectors, detents, snaps or catches.

34. The cooling or warming device of claim 31 wherein the projection is removed from attachment to the base near the proximal end of the projection thereby exposing the projection cavity.

35. The cooling or warming device of claim 34 wherein the proximal end of the projection is attached to the base by attachment means chosen from the group consisting of screw threads, friction fit, hook and loop connectors, detents, snaps or catches.

36. The cooling or warming device of claim 31 wherein the distal end of the projection is removed from proximal end of the projection between the distal end and the proximal end of the projection thereby exposing the projection cavity.

37. The cooling or warming device of claim 36 wherein the proximal end of the projection is attached to the distal end of the projection by attachment means chosen from the group consisting of screw threads, friction fit, hook and loop connectors, detents, snaps or catches.

38. The cooling or warming device of claim 30 wherein the projection outer surface includes a projection access door allowing access to the projection cavity.

39. The cooling or warming device of claim 38 wherein the projection access door is removable from the projection.

40. The cooling or warming device of claim 39 wherein the projection access door is removably attached to the projection outer surface by means selected from the group consisting of a frictional fit, mechanical fit, hook and loop connectors, snaps, catches and detents.

41. The cooling or warming device of claim 39 wherein the projection access door is removably attached to the projection outer surface by a hinge so that the projection access door pivots away from the projection outer surface to provide access to the inner projection cavity.

42. The cooling or warming device of claim 1 wherein the base has:
a base outer surface made of a hard, smooth plastic forming a base cavity within the base; and
a permanent base inner core filling the base cavity, the permanent base inner core made of a material having a high heat index.

43. The cooling or warming device of claim 42 wherein the permanent base inner core is made of a material chosen from a group consisting of polyvinyl chlorides (PVC) or Metallocene polyethylene (MPE) or ethylene-vinyl acetate (EVA).

44. The cooling or warming device of claim 1 wherein the base has:
a base outer surface made of a hard, smooth plastic forming a base cavity within the base; and
a removable base inner core filling the base cavity, the removable base inner core made of a material having a high heat index.

45. The cooling or warming device of claim 44 wherein the removable base inner core is made of a material chosen from a group consisting of polyvinyl chlorides (PVC) or Metallocene polyethylene (MPE) or ethylene-vinyl acetate (EVA).

46. The cooling or warming device of claim 1 wherein the base has a base outer surface made of a hard, smooth plastic forming a base cavity within the base.

47. The cooling or warming device of claim 46 wherein access to the base cavity is provided by removing a portion of the base outer surface.

48. The cooling or warming device of claim 47 wherein the bottom end of the base is removed thereby exposing the base cavity.

49. The cooling or warming device of claim 48 wherein the bottom end of the base is attached to the remainder of the base by attachment means chosen from the group consisting of screw threads, friction fit, hook and loop connectors, detents, snaps or catches.

50. The cooling or warming device of claim 47 wherein the top end of the base is removed thereby exposing the base cavity.

51. The cooling or warming device of claim 50 wherein the top end of the base is attached to the remainder of the base by attachment means chosen from the group consisting of screw threads, friction fit, hook and loop connectors, detents, snaps or catches.

52. The cooling or warming device of claim 46 wherein the base surface includes a base access door allowing access to the base cavity.

53. The cooling or warming device of claim 52 wherein the base access door is removable from the base.

54. The cooling or warming device of claim 53 wherein the base access door is removably attached to the base outer surface by means selected from the group consisting of a frictional fit, mechanical fit, hook and loop connectors, snaps, catches and detents.

55. The cooling or warming device of claim 53 wherein the base access door is removably attached to the base outer surface by a hinge so that the base access door pivots away from the base outer surface to provide access to the base cavity.

56. The cooling or warming device of claim 1 wherein the base has and outer edge and a back surface and wherein the back surface is separated from the remainder of the base along the outer edge.

57. The cooling or warming device of claim 56 wherein the back surface of the base is attached to the base by means chosen from the group consisting of screw threads, friction fit, hook and loop connectors, detents, snaps or catches.

58. The cooling or warming device of claim 1 further comprising
a head unit comprising a first junction of a Peltier circuit, the head unit located in functional contact with either the projection or the base;
an end unit comprising a second junction of a Peltier circuit;
a power source to provide power to the Peltier circuit, the power source connected to the head unit by a first connection wire and to the end unit by a second connection wire.

59. The cooling or warming device of claim 58 further comprising a switch connected between the power source and either the head unit or end unit or both to control the operation of the Peltier circuit.

60. The cooling or warming device of claim 58 wherein the head unit is located within and in functional contact with the projection.

61. The cooling or warming device of claim 58 wherein the head unit is located on the contact surface of the base and wherein the cooling or warming device has a projection heat sink in contact with head unit extending into the projection.

62. The cooling or warming device of claim 58 wherein the head unit is located within and in functional contact with the base.

63. The cooling or warming device of claim 58 further comprising
a second head unit comprising a first junction of a Peltier circuit, the second head unit located in functional contact with the projection when the head unit is located in functional contact with the base or in functional contact with the base when the head unit is located in functional contact with the projection; and
a second end unit comprising a second junction of a Peltier circuit.

64. The cooling or warming device of claim 58 wherein the end unit is located on the back surface of the base.

65. The cooling or warming device of claim 58 wherein the power source is a battery.

66. The cooling or warming device of claim 58 wherein the power source receives power from common household electrical current.

67. The cooling or warming device of claim 1 further comprising a container for containing the cooling or warming device.

68. The cooling or warming device of claim 67 wherein the container is a box with a body and a lid.

69. The cooling or warming device of claim 68 wherein the lid is attached to the body by a hinge.

70. The cooling or warming device of claim 68 wherein the lid is attached to the body by means chosen from the group consisting of screw threads, friction fit, hook and loop connectors, detents, snaps or catches.

71. The cooling or warming device of claim 1 further comprising a therapeutic jelly.

72. The cooling or warming device of claim 71 wherein the therapeutic jelly is a lubricant.

73. The cooling or warming device of claim 1 further comprising a condom for covering the projection.

74. A cooling or warming device comprising:
a base, the base having a contact surface, a back surface, a bottom end, a top end,
a base outer surface and an outer edge that extends around the periphery of the base, the contact surface having a ridge running from the projection toward the top end;
an elongated projection having a distal end and a proximal end and a projection outer surface, the projection attached to base at the proximal end;
wherein the cooling or warming device is made substantially of a material of device 10 having a high heat capacity whereby once the cooling or warming device is cooled or warmed prior to use, the cooling or warming device will remain cool or warm, respectively, through use of the cooling or warming device.

75. A cooling or warming device comprising:
a base, the base having a contact surface, a back surface, a bottom end, a top end, a base outer surface and an outer edge that extends around the periphery of the base, the contact surface having a ridge running from the projection toward the top end;
an elongated projection having a distal end and a proximal end and a projection outer surface, the projection attached to base at the proximal end, wherein the projection has:
a projection outer surface made of a hard, smooth plastic forming a projection cavity within the projection; and
a permanent projection inner core filling the projection cavity, the permanent inner core made of a material having a high heat index.

76. A cooling or warming device comprising:
a base, the base having a contact surface, a back surface, a bottom end, a top end, a base outer surface and an outer edge that extends around the periphery of the base, the contact surface having a ridge running from the projection toward the top end;
an elongated projection having a distal end and a proximal end and a projection outer surface, the projection attached to base at the proximal end, wherein the projection has:
a projection outer surface made of a hard, smooth plastic forming a projection cavity within the projection; and
a removable inner core filling the projection cavity, the removable inner core made of a material having a high heat index.

77. A cooling or warming device comprising:
a base, the base having a contact surface, a back surface, a bottom end, a top end, a base outer surface and an outer edge that extends around the periphery of the base, the contact surface having a ridge running from the projection toward the top end;
an elongated projection having a distal end and a proximal end and a projection outer surface, the projection attached to base at the proximal end, wherein the projection has a projection outer surface made of a hard, smooth plastic forming a projection cavity within the projection.

78. A cooling or warming device comprising an elongated projection adapted to be placed into with a user's vagina wherein the projection base is made substantially of a material having a high heat capacity whereby once the cooling or warming device is cooled or warmed prior to use, the cooling or warming device will remain cool or warm, respectively, through use of the cooling or warming device.

79. A cooling or warming device comprising a base having a contact surface adapted to come into contact with a user's genitals, a back surface, a bottom end, a top end, a base outer surface, an outer edge that extends around the periphery of the base and a ridge running from the projection toward the top end, the ridge having a peak that is displaced from the contact surface of the base and wherein the ridge has a low height at its peak that tapers to near the outer edge, and wherein the base is made substantially of a material having a high heat capacity whereby once the cooling or warming device is cooled or warmed prior to use, the cooling or warming device will remain cool or warm through use of the cooling or warming device.

80. A method of treating inflammation or irritation to a woman's genitals comprising the steps of:
providing a cooling or warming device comprising:
a base;
an elongated projection having a distal end and a proximal end and a projection outer surface, the projection attached to base at the proximal end;
cooling or warming the cooling or warming device; and
placing the cooling or warming device in contact with a user's genitals.

81. The method of treating inflammation or irritation to a woman's genitals of claim 80 wherein the step of cooling or warming the cooling or warming device includes the steps of:
putting the cooling or warming device in a cool or cold or warm environment; and
letting the cooling or warming device cool or warm, respectively, to the desired temperature.

82. The method of treating inflammation or irritation to a woman's genitals of claim 80 wherein the step of cooling or warming the cooling or warming device includes the step of cooling the cooling or warming device to a temperature between about 15° F. to about 50° F.

83. The method of treating inflammation or irritation to a woman's genitals of claim 81 wherein the step of cooling the cooling or warming device to a temperature between about 15° F. to about 50° F. includes the step of cooling the cooling or warming device to a temperature of about 25° F.

84. The method of treating inflammation or irritation to a woman's genitals of claim 80 wherein the step of cooling or warming the cooling or warming device includes the step of warming the cooling or warming device to a temperature between about 97° F. to about 110° F.

85. The method of treating inflammation or irritation to a woman's genitals of claim 83 wherein the step of warming the cooling or warming device to a temperature between about 97° F. to about 110° F. includes the step of warming the cooling or warming device to a temperature between about 100° F. to about 105° F.

86. The method of treating inflammation or irritation to a woman's genitals of claim 80 wherein the step of providing a cooling or warming device includes the step of:
providing a projection outer surface made of a hard, smooth plastic forming a projection cavity within the projection; and
a removable projection inner core filling the projection cavity, the removable projection inner core made of a material having a high heat index; and
wherein the step of cooling or warming the cooling or warming device includes the steps of:
removing the projection inner core from the projection cavity;
cooling or warming the projection inner core;
reinserting the projection inner core in the projection cavity; and
allowing the cooled or warmed projection inner core to cool or warm, respectively, the projection.

87. The method of treating inflammation or irritation to a woman's genitals of claim 80 wherein:
the step of providing a cooling or warming device includes the step of:
providing a base having a base outer surface made of a hard, smooth plastic forming a base cavity within the base; and
a removable base inner core filling the base cavity, the removable base inner core made of a material having a high heat index; and
the step of cooling or warming the cooling or warming device includes the steps of:
removing the base inner core from the base cavity;
cooling or warming the base inner core;
reinserting the base inner core in the base cavity; and
allowing the cooled or warmed base inner core to cool or warm, respectively, the base.

88. The method of treating inflammation or irritation to a woman's genitals of claim 80 wherein:
the step of providing a cooling or warming device includes the step of:
providing an elongated projection having a projection outer surface made of a hard, smooth plastic forming a projection cavity within the projection;
the step of cooling the cooling or warming device includes the steps of:
placing ice, cold water or both into the projection cavity;
allowing the ice, cold water or both to cool the projection.

89. The method of treating inflammation or irritation to a woman's genitals of claim 80 wherein:
the step of providing a cooling or warming device includes the step of:
providing an elongated projection having a projection outer surface made of a hard, smooth plastic forming a projection cavity within the projection;
the step of warming the cooling or warming device includes the steps of:
placing warm water or other warm liquid into the projection cavity;
allowing the warm water or other warm liquid to warm the projection.

90. The method of treating inflammation or irritation to a woman's genitals of claim 80 wherein:
the step of providing a cooling or warming device includes the step of:
providing a base having a base outer surface made of a hard, smooth plastic forming a base cavity within the base;
the step of cooling the cooler includes the steps of:
placing ice, cold water or both into the base cavity;
allowing the ice, cold water or both to cool the base.

91. The method of treating inflammation or irritation to a woman's genitals of claim 80 wherein:
the step of providing a cooling or warming device includes the step of:
providing a base having a base outer surface made of a hard, smooth plastic forming a base cavity within the base;
the step of warming the cooler includes the steps of:
placing warm water or other warm liquid into the base cavity;
allowing the warm water or other warm liquid to warm the base.

92. The method of treating inflammation or irritation to a woman's genitals of claim 80 wherein:
the step of providing a cooling or warming device includes the step of providing:
a head unit comprising a first junction of a Peltier circuit, the head unit located in functional contact with either the projection or the base;
an end unit comprising a second junction of a Peltier circuit;
a power source to provide power to the Peltier circuit, the power source connected to the head unit by a first connection wire and to the end unit by a second connection wire; and
the step of cooling or warming the cooling or warming device includes the steps of:
activating the Peltier circuit to cool or warm, respectively, the cooling or warming device.

93. A method of treating vaginitis comprising the steps of:
providing a cooling or warming device comprising:
a base;
an elongated projection having a distal end and a proximal end and a projection outer surface, the projection attached to base at the proximal end;
cooling or warming the cooling or warming device; and
placing the cooling or warming device in contact with a user's genitals.

94. A method of treating inflamed vaginal and vulvar tissue resulting from trauma or irritation comprising the steps of:
providing a cooling or warming device comprising:
a base;
an elongated projection having a distal end and a proximal end and a projection outer surface, the projection attached to base at the proximal end;
cooling or warming the cooling or warming device; and
placing the cooling or warming device in contact with a user's genitals.

95. A method of treating inflamed vaginal and vulvar tissue resulting from sexual activity with inadequate vaginal lubrication especially in post menopausal women and from sexual activity for post menopausal women who have decreased elasticity in their vaginal tissues comprising the steps of:
providing a cooling or warming device comprising:
a base;
an elongated projection having a distal end and a proximal end and a projection outer surface, the projection attached to base at the proximal end;
cooling or warming the cooling or warming device; and
placing the cooling or warming device in contact with a user's genitals.

96. A method of treating inflamed vaginal and vulvar tissue resulting from increased sexual activity comprising the steps of:
providing a cooling or warming device comprising:
a base;
an elongated projection having a distal end and a proximal end and a projection outer surface, the projection attached to base at the proximal end;
cooling or warming the cooling or warming device; and
placing the cooling or warming device in contact with a user's genitals.

97. A method of treating inflamed vaginal and vulvar tissue in post-partum women resulting from trauma or irritation from the childbirth process comprising the steps of:
providing a cooling or warming device comprising:
a base;
an elongated projection having a distal end and a proximal end and a projection outer surface, the projection attached to base at the proximal end;
cooling or warming the cooling or warming device; and
placing the cooling or warming device in contact with a user's genitals.

98. A method of treating inflamed vaginal and vulvar tissue in non-human animals resulting from trauma or irritation comprising the steps of:
providing a cooling or warming device comprising:
a base;
an elongated projection having a distal end and a proximal end and a projection outer surface, the projection attached to base at the proximal end;
cooling or warming the cooling or warming device; and
placing the cooling or warming device in contact with a animal's genitals.
